(19) United States
(12) Patent Application Publication
(10) Pub. No.: US 2016/0000514 A1
(43) Pub. Date: Jan. 7, 2016

(54) SURGICAL VISION AND SENSOR SYSTEM

(71) Applicant: Alan Ellman, Hewlett, NY (US)

(72) Inventor: Alan Ellman, Hewlett, NY (US)

(21) Appl. No.: 14/748,212

(22) Filed: Jun. 23, 2015

Related U.S. Application Data
(60) Provisional application No. 62/020,420, filed on Jul. 3, 2014.

(51) Int. Cl. A61B 19/00

(52) U.S. Cl. CPC A61B 19/26; A61B 19/50; A61B 19/5212; A61B 2019/262; A61B 2019/507; A61B 2576/02

(57) ABSTRACT

The present invention pertains to an enhanced surgical tool and display device for conducting surgical procedures which includes a wearable headgear including a frame for positioning an optical display into a field of view. A controller is attached to a portion of the frame and adapted to provide a computer generated image to an optical display. An optical display, including an active side for projecting a computer generated image, electrically communicates with the controller and is attached to the controller such that the active side is visible in a field of view.

### RELATED APPLICATIONS

This application claims priority to Provisional Patent Application No. 62/020,420, the entirety of which is incorporated herein by reference.

### FIELD OF INVENTION

The present invention relates to an enhanced surgical tool in combination with a surgical display headgear apparatus.

### BACKGROUND

Surgical procedures, particularly those involving the human spine, require extreme attention to detail. A surgeon desires to manipulate portions of the body's tissues in the most specific and sensitive manner, impacting only the body portions that are desired to be affected and minimally affecting healthy body tissue. Current technology for surgical tools is, at times, not as finely tuned as would be desired to selectively cut, eliminate, or repair damaged or diseased tissue while maneuvering around healthy body tissue and sensitive tissues such as nerve roots. This is, in part, because of the very small scaling of the body parts on which to be operated. The operative site can be difficult for a surgeon to see because it may be located far into the interior of the body and because the surgical field of view can be obstructed by the surgeon's own hand or by portions of the patient's body. Visual images and navigational maps of a patient's body can provide knowledge of the positioning of a surgical instrument in the surgical site and can assist a surgeon in performing a more optimal surgical procedure.

### SUMMARY OF THE INVENTION

The present invention pertains to an enhanced surgical tool and display device for conducting surgical procedures which includes a wearable headgear including a frame for positioning an optical display into a field of view. A controller is attached to a portion of the frame and adapted to provide a computer generated image to an optical display. An optical display, including an active side for projecting a computer generated image, electrically communicates with the controller and is attached to the controller such that the active side is visible in a field of view. A surgical tool with an end for grasping the tool and a second distal end for surgical contact with a region of surgically operative material includes a camera attached to the second distal end of the surgical tool and in wireless communication with the controller. The camera is adapted to record and transmit visual images from a surgical site adjacent the distal end of the surgical tool to the controller. The controller is adapted to configure a visual image from the camera for projection to the active side of the optical display.

### BRIEF DESCRIPTION OF THE DRAWINGS

FIG. 1 is a perspective view of surgical display headgear according to an aspect of the present invention;

FIG. 1A is a block diagram of an embodiment of the invention;

FIG. 2 is a perspective view of an enhanced surgical tool according to an aspect of the present invention;

FIG. 3 is a detail view of a portion of enhanced surgical tool of FIG. 2;

FIG. 4A is a perspective view of surgical display headgear according to an aspect of the present invention;

FIG. 4B is a perspective view of surgical display headgear according to an aspect of the present invention;

FIG. 4C is a perspective view of surgical display headgear according to an aspect of the present invention;

FIG. 5 illustrates surgical display headgear of FIG. 4B from another perspective view according to an aspect of the present invention;

FIG. 5A is a perspective view of an above-eye display module according to an aspect of the present invention;

FIG. 5B is a perspective view of an above-eye display module according to an aspect of the present invention;

FIG. 6 is a detail view of a portion of enhanced surgical tool of FIG. 3;

FIG. 7 is a detail view of a portion of enhanced surgical tool of FIG. 3;

FIG. 8A is a side view of a camera according to an aspect of the present invention;

FIG. 8B is a perspective view of a portion of an enhanced surgical tool according to an aspect of the invention

FIG. 8C is a perspective view of a portion of an enhanced surgical tool according to an aspect of the invention;

FIG. 8D is a perspective view of a portion of an enhanced surgical tool according to an aspect of the invention;

FIG. 9 is a block diagram of an embodiment of the invention;

FIG. 10 is a block diagram of an embodiment of the invention;

FIG. 11 is a perspective view an enhanced surgical tool according to an aspect of the invention;

FIG. 12A is a detail view of a portion of enhanced surgical tool of FIG. 11;

FIG. 12B is a detail view of a portion of enhanced surgical tool of FIG. 11;

FIG. 12C is a perspective view an enhanced surgical tool according to an aspect of the invention;

FIG. 12D is a perspective view an enhanced surgical tool according to an aspect of the invention;

FIG. 13 is a perspective view of a guide wire surgical tool according to an aspect of the invention;

FIG. 14 is a detail view of a portion of guide wire surgical tool of FIG. 13;

FIG. 15 is a perspective view of an enhanced surgical tool according to an aspect of the invention;

FIG. 16 is a perspective view of an enhanced surgical tool according to an aspect of the invention;

FIG. 16A is a detail view of a portion of an enhanced surgical tool of FIG. 16;

FIG. 17 is a perspective view of an enhanced surgical tool according to an aspect of the invention;

FIG. 18 is a perspective view of an enhanced surgical tool for suction according to an aspect of the invention;

FIG. 19 is a perspective view of an enhanced surgical tool according to an aspect of the invention;

FIG. 20 is a perspective view of a tissue retractor surgical tool according to an aspect of the invention;

FIG. 21 is a perspective view of a tissue retractor surgical tool according to an aspect of the invention;

FIG. 22 is a perspective view of a scalpel surgical tool according to an aspect of the invention;

FIG. 22A is a close up view of 22A in FIG. 22;

FIG. 23 is a perspective view of a surgical scissors according to an aspect of the invention;

FIG. 24 is a perspective view of a portion of a human face and an enhanced surgical tool according to an aspect of the present invention;

FIG. 25 is a detailed view of a portion of FIG. 24 according to an aspect of the present invention;

FIG. 26 is a perspective view of an above-eye display module according to an aspect of the present invention;

FIG. 27 is a top view of a human torso and a positioning system according to an aspect of the present invention;

FIG. 28 is a side view of a human torso and a positioning system according to an aspect of the present invention

FIG. 29A is a side view of an enhanced surgical tool inserted into tissue according to an aspect of the present invention;

FIG. 29B is a side view of an enhanced surgical tool inserted into tissue according to an aspect of the present invention.

### DETAILED DESCRIPTION

U.S. Pat. No. 7,101,370 B2 entitled Disposable Electrosurgical Handpiece for Treating Tissue, U.S. Pat. No. 7,137,982 B2 entitled RF Electrosurgical Probe, U.S. Pat. No. 7,507,232 B1 entitled Flexible Electrosurgical Electrode with Manipulator, U.S. Pat. No. 7,905,882 B1 entitled Activator for Electrosurgical Handpiece, and U.S. Pat. No. 7,951,146 B2 entitled RF Intervertebral Electrosurgical Probe, the entirety of all of the aforementioned patents are hereby incorporated by reference.

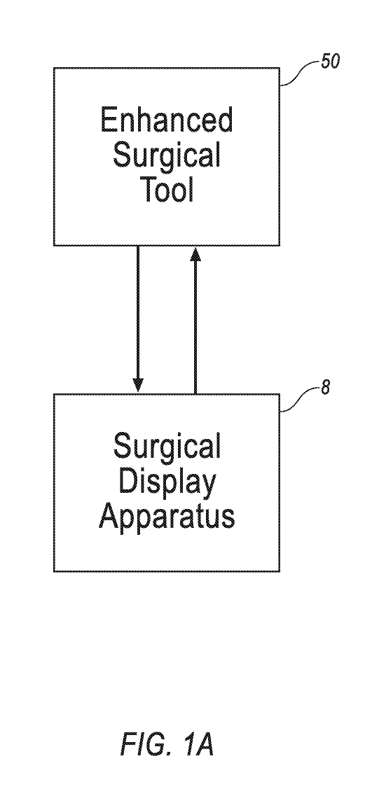

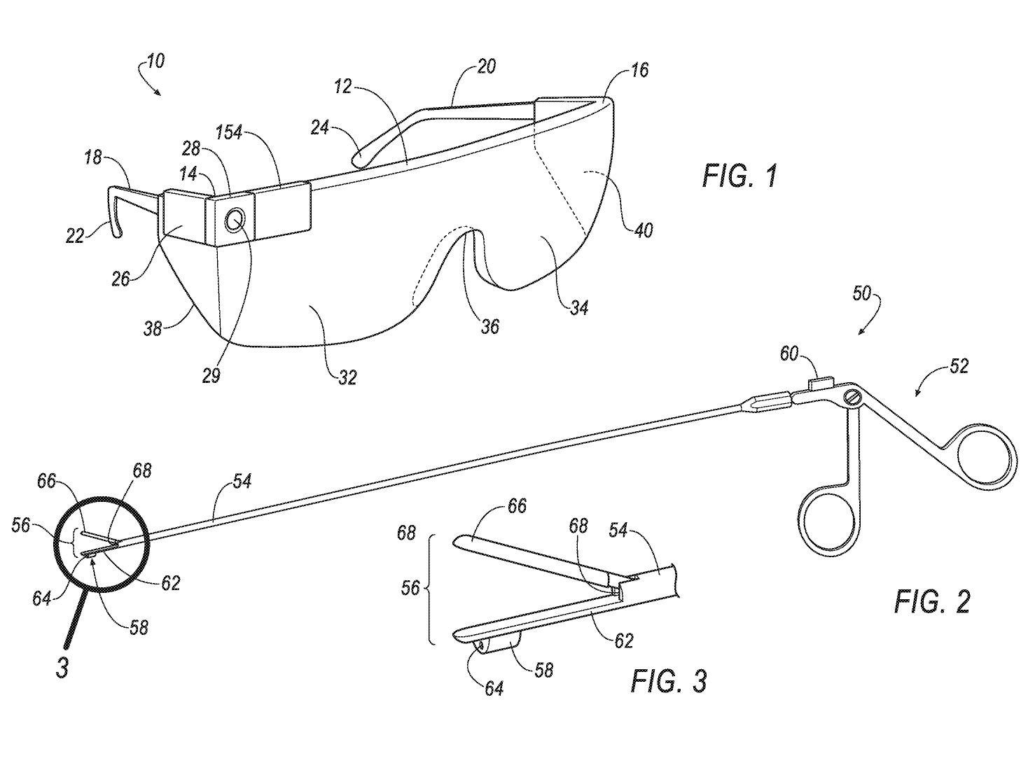

Referring to FIG. 1A, an embodiment of the invention is described in a block diagram. Enhanced surgical tool 50 is in electrical communication with surgical display apparatus 8. Enhanced surgical tool 50 is comprised of any type of surgical tool which may be utilized to perform a surgical operation and further comprises a camera and sensor system providing enhanced functionality which will be described in further detail below. Surgical display apparatus 8, in one embodiment, is a wearable surgical display headgear 10 which interacts with enhanced surgical tool 50 to provide control and display functionality for enhanced surgical tool 50 as will also be described below. In another embodiment, surgical display apparatus 8 is a display such as a computer or TV display that provides the images and information as described below.

Referring now to FIG. 1, an embodiment of surgical display headgear 10 is shown and described. In FIG. 1, surgical display headgear 10 is a wearable apparatus generally shaped as eye-glass frames or a headband structure to support optical elements in a visual field of view. In this aspect of the invention, surgical display headgear 10 comprises forehead bar 12 to be worn in the front of a wearer's head and curving backward at angled portions 14 and 16 to fit around the forehead of a wearer. Forehead bar 12 connects backward to right temple bar 18 and left temple bar 20 ending respectively at right earpiece 22 and left earpiece 24. In this embodiment of the invention, forehead bar 12 supports right lens 32 and left lens 34 and nose piece 36 which can be balanced upon the wearer's nose. Lenses 32 and 34, pictured as one continuous lens, but they may be two, are optional as the invention may be configured without them. If included, lenses 32 and 34 may be comprised of clear, optical quality glass or plastic which may be impact resistant, luminescent, designed as prescriptive eyewear or designed as any other configuration that might aid a surgeon. Optional side shields 38 and 40 may provide protection to the eye from debris. As will be discussed further, surgical display headgear 10 is not limited to an eye-glass type frame design and any other wearable head-mounted apparatus may be employed.

Continuing in reference to FIG. 1 Forehead bar 12 supports headgear controller 26 which is positioned along right temple bar 18 and adjacent angled portion 14. Headgear controller 26 contains circuitry and components for the function of the surgical display headgear 10 which may include a CPU and storage, wireless transmission and receiving circuitry, memory components, sound components, voice recognition components, control firmware and software, a power supply, as well as other circuitry desirable for the functionality of the surgical display headgear 10 and its associated input and output devices. In one embodiment of the invention, headgear controller 26 serves as the activation and connection point for the surgical display headgear 10 connecting it to input devices to receive and transmit commands Headgear controller 26, in one embodiment, is in communication with both headgear camera 28 and above-eye display module 154 and includes circuitry and components for the function of these components. Headgear controller 26 also functions to communicate with other devices such an internet connection, an external computer or CPU, a cellphone, an external display, external audio components or alarm system or a printing device.

Continuing in reference to FIG. 1 Adjacent headgear controller 26 and supported from forehead bar 12 is headgear camera 28. Headgear camera 28 is a miniature camera system or nano-camera of a small, light-weight camera which, in this embodiment, can be activated to provide a visual recording or images of the surroundings of the person wearing the surgical display headgear 10 from the vantage point of their forehead, or focus in on a specific portion of the surgical operation site. Headgear camera 28 may be comprised of a CMOS sensor, a back illuminated digital image sensor, a fiber-optic type camera or other type of camera capable of video recording, video streaming and image taking. The camera may show images through infrared . . . . Headgear camera 28 may be formed within headgear controller 26 housing or be comprised of a separate unit from headgear controller 26. Headgear camera 28 is optional to the present invention. Headgear camera 28 may be positioned facing a forward region of the surgical display headgear 10 and includes a lens 29, which may be a photo lens or optic sensor, facing outward toward subject matter which the wearer would be observing during usage. Headgear camera 28 and headgear controller 26 are attached to forehead bar 12 and are shaped to fit in a curved formation around right angled portion 14 of forehead bar 12.

Continuing in reference to FIG. 1, surgical display headgear 10 includes components capable of projecting a computer generated image (CGI) to above-eye display module 154 (see FIG. 26), which in one aspect of the invention includes an optical display element positioned near or above a wearer's eye and viewable by the person wearing surgical display headgear 10. In this example of the present invention, above-eye display module 154 is connected to headgear camera 28 and positioned in a field of view of the wearer's right eye. Above-eye display module 154 may be positioned in other places and other fields of view of the person wearing the headgear such as above their left eye or other locations for viewing and display. If headgear camera 28 is not included in the invention, headgear controller 26 may be designed to support above-eye display module 154 into a desirable field of view of the user.

Continuing in reference to FIG. 1 above-eye display module 154 may be comprised of an optical display element in conjunction with a system of light projection sources such as lasers, lenses, beam splitters, and other types of projection apparatus designed to project a real-time CGI display on to an active side of a display module 154 is, in one embodiment, in a field of view of the user. Above-eye display module 154 may utilize technology as liquid crystal on silicon (LcoS) for near-eye displays or any projection technology as may be used in other head-mounted display-type applications. Above-eye display module 154 may appear as a prism and be transparent and glass-like to the user, as in the form of an optical quality glass material, when no image is displayed. When an image is sent to above-eye display module 154, it may appear in the form of a text display message, real time video images or still pictures images, 3-D maps, alarm lights and messages with flashing text or colored icons, and any other information that can be formed into a visual display.

Referring now to FIG. 2, one embodiment of enhanced surgical tool 50 is shown and described. In FIG. 2, enhanced surgical tool 50 provides functionality of a particular surgical tool and is in wireless or wired communication with the surgical display headgear 10. In this embodiment of the invention, enhanced surgical tool 50 may be used to perform a surgical function such as a discectomy procedure typical of the Elliquence Disc-FX surgical tool. It should be understood and will be discussed further that other types of surgical tools, some which may be well-known in the art, may be utilized as will be illustrated in other embodiments of the invention. In this embodiment, enhanced surgical tool 50 includes in-situ camera and sensor system 58 which is located in close proximity to the active end 56 of enhanced surgical tool 50. In the present invention, in-situ camera and sensor system 58 may include a camera, sensors, light sources, a positioning circuitry and other components as will be described in further detail in the following paragraphs. In-situ camera 64 of in-situ camera and sensor system 58 is a miniature, nano-sized camera which is capable of recording, photographing, video recording, video streaming and transmitting visual information. In this example, in-situ camera and sensor system 58 is comprised of a fiber optic type camera or fiberscope with a wide angle lens; however, it is understood that CMOS sensor, back illuminated digital image sensor, or other types of cameras or optical recording systems may be employed. In-situ camera and sensor system 58 may include positioning circuitry (not shown) for detection of a specific portion of enhanced surgical tool 50 such as active end 56 in a context similar to a Global Positioning System (GPS) which is described in further detail below.

Continuing with an embodiment of the invention shown in FIG. 2, enhanced surgical tool 50 includes surgical tool controller 60 which is connected to in-situ camera and sensor system 58 by way of fiber optic or electrical connection via a conduit through extension portion 54. Surgical tool controller 60 may alternatively be included with in-situ camera and sensor system 58 and be located within or adjacent to in-situ camera and sensor system 58. Surgical tool controller 60 provides wireless or wired communication from in-situ camera and sensor system 58 to headgear controller 26 and, optionally, to other external devices such as a computer or an external display. In-situ camera and sensor system 58 may include circuitry for wireless communication with other devices such as headgear controller 26 or surgical tool controller 60. Optionally, in-situ camera and sensor system 58 may include circuitry for wireless communication to headgear controller 26 directly, rather than to surgical tool controller 60. In such an embodiment, the components of surgical tool controller 60 may be contained within headgear controller 26.

FIG. 3 is a magnified view of 3 in FIG. 2 which illustrates a detailed perspective view of an aspect of the invention in which in-situ camera and sensor system 58 is positioned on the lower surface 62 of extension portion 54 and wherein lens of camera 64 is directed toward the active end 56 (i.e. the most distal end) of enhanced surgical tool 50 away from the gripping mechanism 52 as held by a user of the tool. In-situ camera and sensor system 58 may be positioned at any number of different locations on enhanced surgical tool 50. The position of in-situ camera and sensor system 58 may be selected to best focus on the activities being performed at a surgical site. For example, in-situ camera and sensor system 58 may be positioned on the upper surface 66 of extension portion 54 or within the hinge apparatus 68. Multiple in-situ cameras may also be utilized. Methods of construction and/or attachment of camera and sensor system 58 to enhanced surgical tool 50 are discussed in further paragraphs.

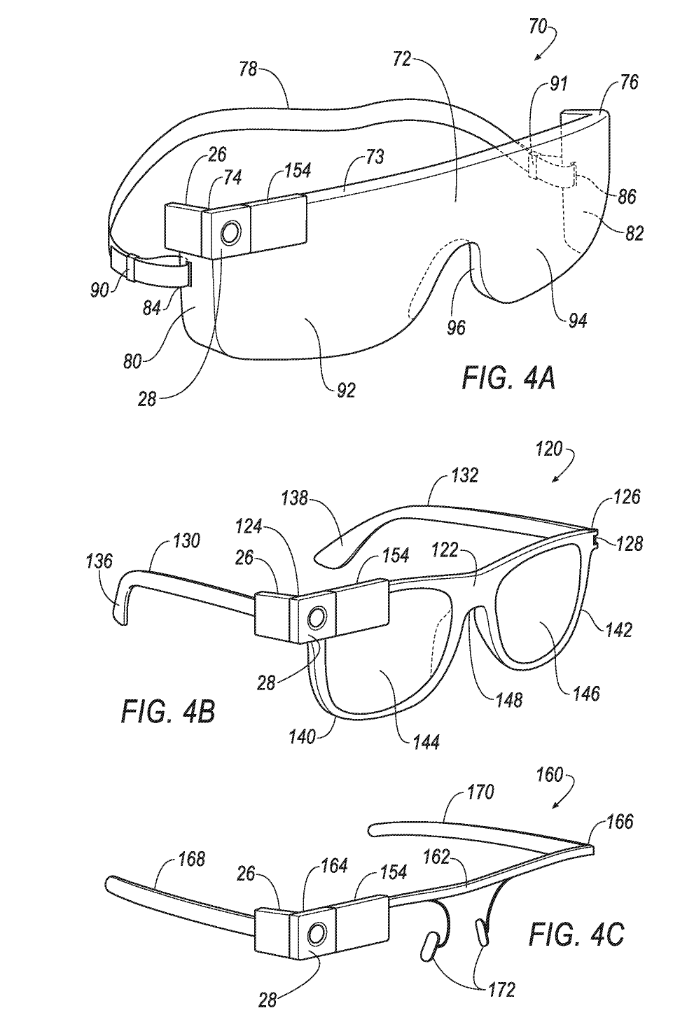

In reference to FIGS. 4A through 4C, several embodiments of surgical display headgear are shown. In FIG. 4A, surgical display headgear 70 includes eye-cover 72 which may be a singularly formed glass or plastic cover for eye protection which includes an upper edge 73 that abuts a forehead when worn, two lower lens-shaped portions, right lens 92 and left lens 94, and nosepiece 96. Eye-cover 72 bends back at angled portions 74 and 76 to connect to right side shield 80 and left side shield 82 through which a strap 78 is fastened. In this example, eye shields 80 and 82 each include slots 84 and 86 through which strap 78 is drawn and fastened back with adjustable clips 90 and 91. In this embodiment, headgear controller 26, headgear camera 28, and above-eye display module 154 are attached to eye-cover 72 at the upper edge 73 above right lens-shaped portion 92 so that the eye-cover 72 and the adjustable strap 78 hold above-eye display module 154 above the right eye of a wearer.

FIG. 4B illustrate an aspect of the invention in which surgical display headgear 120 comprises traditional eyeglass frame 122 securing right eyeglass lens 144 and left eyeglass lens 146 into respective lens holder 140 and 142. In this example, eyeglass frame 122 is connected by a hinges 128 and 124 to right temple arm 130 and left temple arm 132 which end in ear pieces 136 and 138 used to secure surgical display headgear 120 to a user's head. Headgear camera 28 and headgear controller 26 are located on the upper right side of right lens 144 and attached to eyeglass frame 122. Above-eye display module 154 is attached to headgear camera 28. As a user positions the surgical display headgear 120 on his or her head with the nose piece 148 atop the bridge of their nose and earpieces 136 and 138 secured behind their ears, above-eye display module 154 is positioned into a field of view above their right eye.

FIG. 4C illustrates surgical display headgear 160 in a minimalist design in which forehead bar 162 has a “wrap around” design and turns backward at angled portions 164 and 166 ending in temple arms 168 and 170. The pressure from temple arms 168 and 170 on a wearer's temple along with the support from nose piece 172 atop a wearer's nose holds the surgical display headgear 160 on the wearer's head. Headgear controller 26 and headgear camera 28 are attached to forehead bar 162 abutting angled portion 164 so that headgear camera 28 supports above-eye display module 154 into a field of view above the wearer's right eye. In yet another embodiment, instead of the headgear that is mountable, the display headgear may take the form of contact lenses. In such an instance, micro implants may be inserted into the contacts such that the contacts display images on a surface of the contacts facing the user's eyes. The contacts may communicate wirelessly with the surgical controller or instruments to provide the images as described in other embodiments.

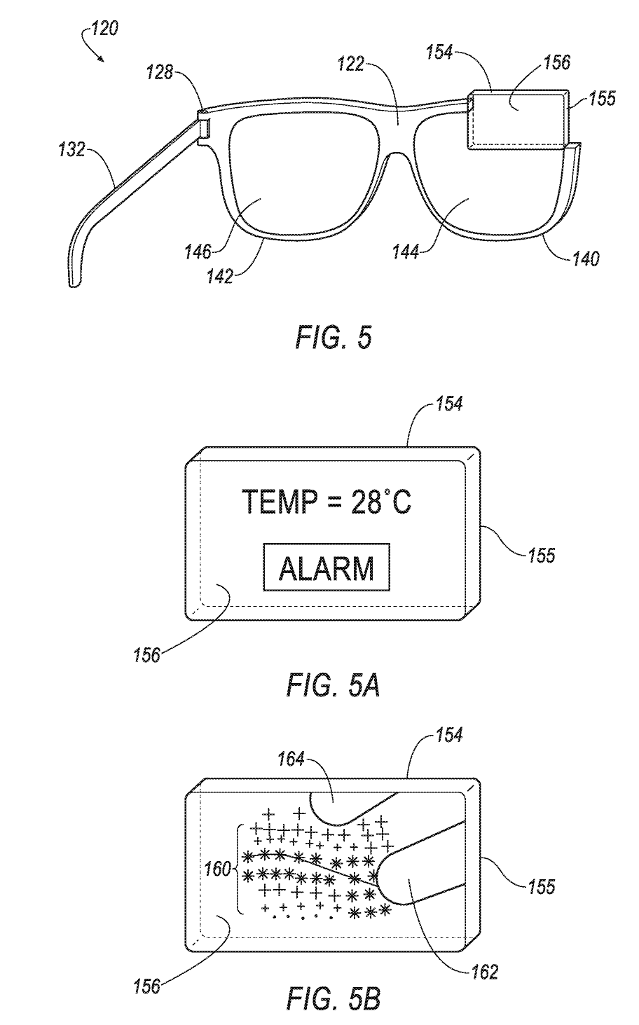

With reference to FIG. 5, a perspective view from the interior of surgical display headgear 120 of FIG. 4 is illustrated. In this example, right temple arm 130, headgear controller 26 and headgear camera 28 are removed to illustrate the position of above-eye display module 154 from the perspective of the wearer. Right edge 155 of above-eye display module 154 would typically be connected to headgear camera 28 in combination with headgear controller 26 to receive input for display on the active side 156 of above-eye display module 154. In the present example, above eye display module 154 has an active side 156 facing a user that provides various information in response to inputs as will be discussed. Display module 154 may be a LCD, LED, transparent or other known display device that displays information in response to input from headgear controller 26 to a user or surgeon. In the present example, above-eye display module 154 is transparent when not in an active mode of computer generated display. FIG. 5A illustrates one example of a detailed view of above-eye display module 154 with an active display of text received from headgear controller 26. The illustration of FIG. 5A shows an indication of temperature as well as an alarm. The temperature may represent a temperature of a patient or operative area and the alarm may represent a point of concern or other issue associated with the surgical procedure. In an aspect of the present invention, FIG. 5B illustrates a view of above-eye display module 154 with an active display of an image of a surgical procedure on a portion of body tissue. In this example, a portion of an enhanced surgical tool 50 including active end sections 162 and 164 is visible adjacent a portion of surgical body tissue 160. The image is displayed on an active side 156 of above-eye display 154. The image may be provided by an in-situ camera through various processing components as will be discussed. A real-time display of activities at the site of the surgical procedure may be sent from enhanced surgical tool 50 to the above-eye display module 154 as will be discussed.

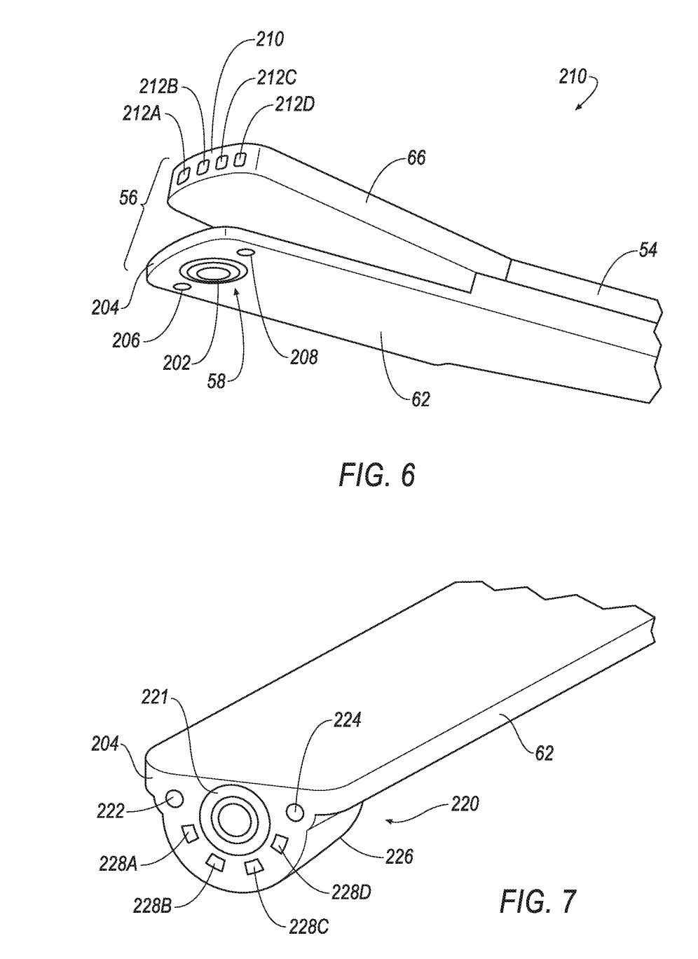

For purposes of description, FIG. 6 illustrates an in-situ camera 202 being embedded in an enhanced surgical tool 50 such that the body of the in-situ camera 202 is positioned within the enhanced surgical tool 50. Likewise, FIG. 7 illustrates an in-situ camera 221 being mounted external to the enhanced surgical tool 50. It will be understood that the embodiments described with respect to FIG. 6 and FIG. 7 may apply to any of the enhanced surgical tools described in various embodiments in the present application.

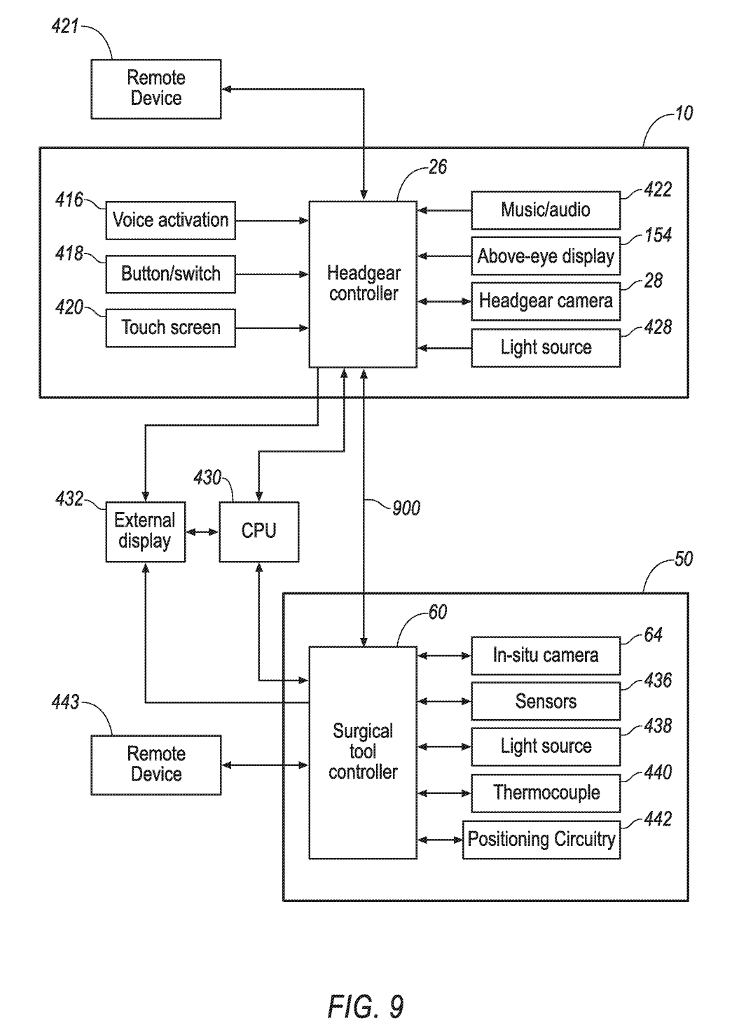

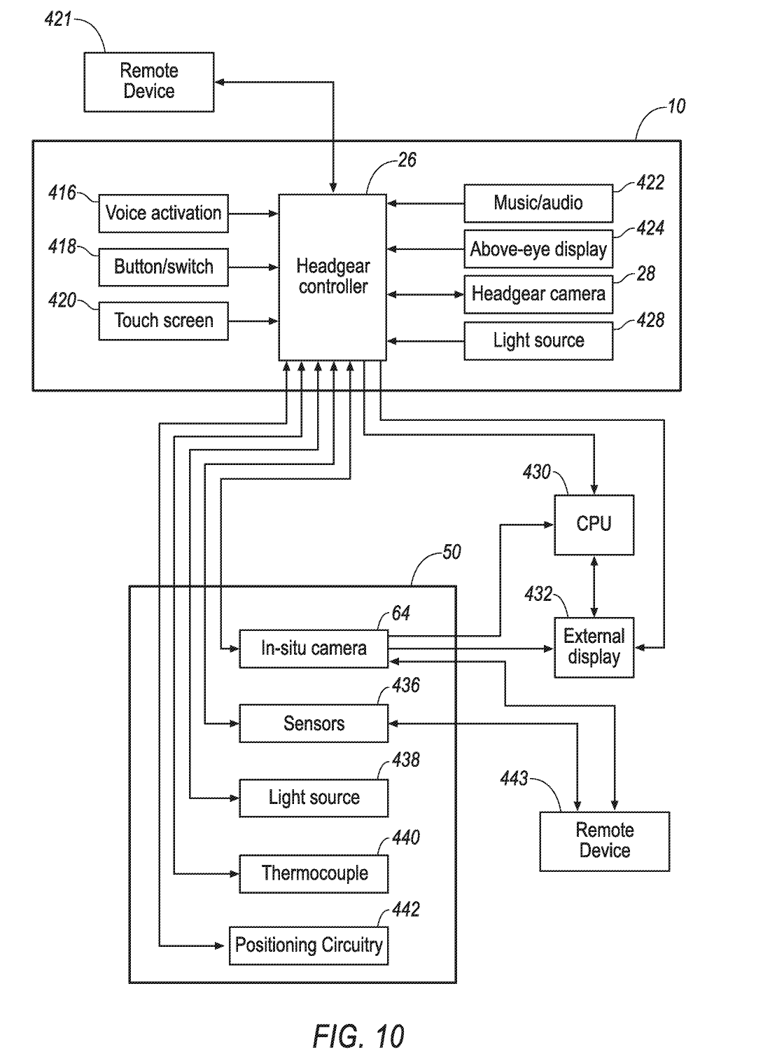

With regard to FIG. 6, a detailed view of an embodiment of the invention shown in FIGS. 2 and 3 is illustrated. FIG. 6 shows a close-up perspective view of the active end 56 of enhanced surgical tool 50. In this example, in-situ camera 202 and light sources 206 and 208 are incorporated into lower surface 62 of enhanced surgical tool 50. These components, in one aspect, are adjacent distal end 204 of lower surface 62 which is located at active end 56 of enhanced surgical tool 50. Additionally, sensors 212A through 212D are incorporated into distal end 210 of upper surface 66, also located at active end 56 of enhanced surgical tool 50. In this example, four sensors 212A through 212D are illustrated, but is should be understood that the number of sensors is an optional feature of the invention and the number may be more or less than is shown in FIG. 6. Each of sensors 212A through 212D, in-situ camera 2020, and light sources 206 and 208 may be in wireless communication with surgical tool controller 60 or headgear controller 26. Alternatively, they may be connected back to surgical tool controller 60 via fiber or electrical connections through a conduit in the interior of extension portion 54 (See FIGS. 9 and 10).

Continuing in reference to FIG. 6, in an aspect of the present invention, the above discussed sensors may be employed to detect operational characteristics of the surgical tool and of the condition of bodily tissues and structures at the surgical site. Types of sensors 212A through 212D may include sensors for heat detection, detection of different spectrums of light including infrared light or UV or other frequencies, sensors to detect electrical characteristics of body tissue such as inductance, power, current, voltage and impedance or radioactive detection for radioactive isotopes using scintigraphy, x-ray, or other radioactive imagery. Additional sensors may be included to detect humidity or moisture composition of tissue, chemical changes of tissue surface due to curing, injecting or addition of medication or detection chemicals. Sensors may include a thermocouple for physical measurement of heat on or below the surface of body tissue. A thermocouple sensor may be extendable or retractable back into the enhanced surgical tool 210 such that a measurement of temperature can be taken at a specific depth below the upper surface of body tissue, liquid or skin and the thermocouple retracted back out of the way of the surgical tool and its operations. It should be understood that many different types of sensors can be included in this invention relating to the characterization of the physical location surrounding and at a surgical site.

FIG. 7 illustrates a close-up perspective view of active end 56 of enhanced surgical tool 50 in which components of in-situ camera and sensor system 220 that includes camera 221, sensors 228A through 228D, and light sources 222 and 224 are incorporated into distal end 204 of lower surface 62 of enhanced surgical tool 50 and external to the device. In this embodiment, lower surface 62 of enhanced surgical tool 50 is constructed such that the components, camera 221, sensors 228A through 228D, and light sources 222 and 224 are built into the material comprising lower surface 62 which may be stainless steel or another surgical quality material. The interior of lower surface 62 may constructed to include a conduit for connection of surgical tool controller 60 to each of these components. Alternatively, surgical tool controller 60 may also be built into the material comprising lower surface 62 and in connection with the components providing electrical communication to the components. Each of sensors 228A through 228D, in-situ camera 221, and light sources 222 and 224 may be in wireless communication with surgical tool controller 60 or headgear controller 26. Alternatively, they may be connected back to surgical tool controller 60 via fiber or electrical connections in the interior of extension portion 54 and not shown in this figure.

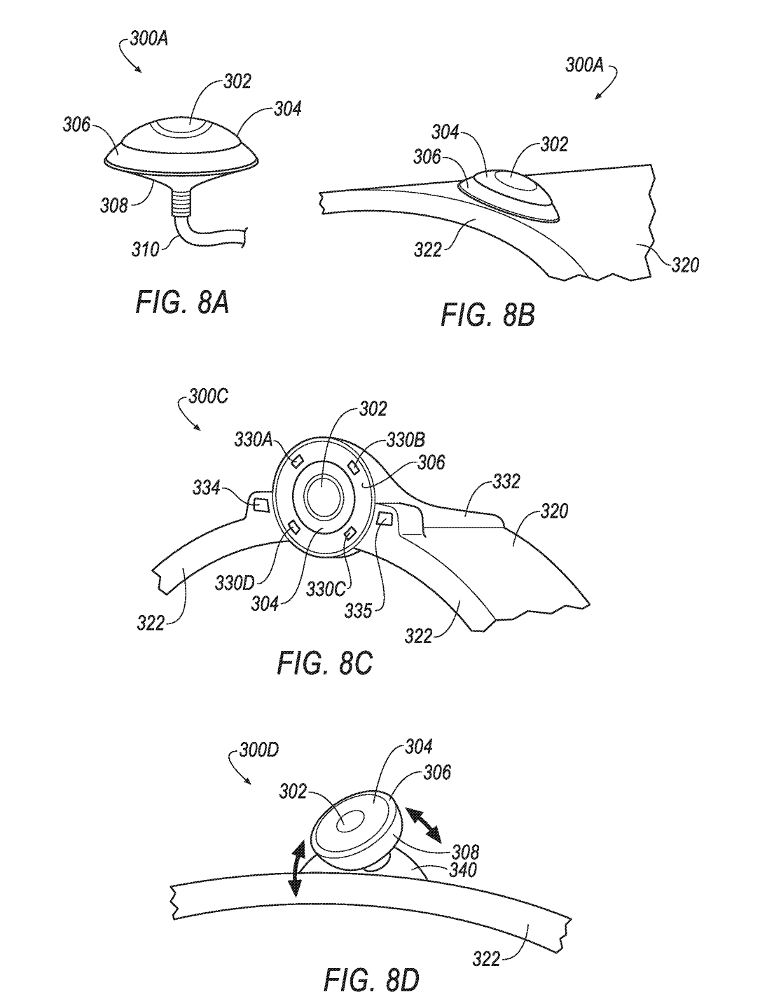

Referring now to FIG. 8A, a side view of a camera 300A according to an aspect of the invention is shown. Camera 300A includes a fish-eye or wide angle type lens 302 with protective cover 304 enclosed within frame 306 and supported by camera housing 308. Camera 300A includes a connection cable 310 which transfers the optical signal of the camera back to a surgical tool controller. The connection cable 310 may be of fiber-optic or standard electrical connection. Alternatively, a wireless connection may be utilized.

Continuing in reference to FIG. 8A, the camera 300A can be mounted within a receiving aperture or hole of enhanced surgical tool 50 (for example lower surface 62 in FIG. 6). More specifically, with reference to FIG. 8B, an example of in-situ camera and sensor system 300A is shown incorporated into an outer surface 320 of a portion of an enhanced surgical tool 50 in which the outer surface 320 is curved or cylindrical or other suitable shape. In-situ camera and sensor system 300A, in this figure, includes a lens 302 under protective cover 304 and secured to outer surface 320 by a connection to frame 306 adjacent an active end 322 of a surgical tool. The housing 308 and cable connection portions 310 of in-situ camera and sensor system 300A are visible in FIG. 8A. In this embodiment, the lens 302 is directed outward from the outer surface. However, the wide optical view of a wide-eye type camera (if one is chosen as the desired camera) captures subject matter near edge 322. Focus, panning and zoom movements of lens of in-situ camera can also capture subject matter near edge 322. The camera may be fitted with a motor to allow more precise viewing angles to potentially be shown.

Referring now to FIG. 8C, in-situ camera and sensor system 300C is positioned at an active edge 322 of a surgical tool with a curved or cylindrical outer surface 320. Lens 302, cover 304 and frame 306 are built into the edge 322 so that lens 302 faces most directly toward the surgical site interacting with active edge 322 in close proximity to the surgical site. In this aspect of the invention, housing and connection cable for in-situ camera and sensor system 300C are enclosed in the surface 320 via a conduit or channel such as a portion of channel structure 332 to connect to surgical tool controller 60. The sensing end of sensors 330A-330D are shown built into frame 306. Electrical or fiber connections to the sensors 330A through 330D are included in channel 332 to connect the sensors 330A-330D to surgical tool controller 60. Light sources 334 and 335 are shown on opposing sides of in-situ camera and sensor system 300C, but may be located on other parts edge 322 of surgical tool. Light sources may be LED type for camera lighting or other types of light sources such as UV or infrared or other frequency. Sensors 330A-330D may include sensors for heat, temperature, light sensors, power sensors for current, voltage, impedance, radioactivity, moisture and humidity sensors, as well as other sensors which may provide feedback information as to the conditions at the site of the surgical procedure. As such, for this or other embodiments, it will be understood that the light sources are optional, for example, where the operative field (for example heat from the patient's body or injected isotopes) emit the radiation or other signals to be detected by the sensors. FIG. 8D includes in-situ camera and sensor system 300D which is shown on a camera mount 340 which is capable of swivel movement to direct camera lens 302 toward the optimal optical field of view for camera lens 302. Such movement may be undertaken through signals provided by surgical tool controller 60 received by the in-situ camera and sensor system 300D through the fiber optic or electrical connections previously discussed. The signals may instruct a motor to move or rotate to redirect the in-situ camera toward a different region to be observed.

FIG. 9 shows a block diagram of an embodiment of the invention. In this example, components of the surgical display headgear 10 communicate with components of the enhanced surgical tool 50 by way of wireless (or wired depending on the embodiment) communication 900 between the headgear controller 26 and the surgical tool controller 60. A person using the invention may input commands to the surgical display headgear 10 though inputs of voice activation circuitry 416, touch activation circuitry 420, buttons and switches 418, each as components of the surgical display headgear 10. The surgical display headgear 10 may also be configured such that the headgear controller is adapted to communicate with a remote device 421 such as a computer or cell phone. Headgear controller 26 is adapted and programmed so that the surgeon or user may direct commands to a music component including a music player, an audio microphone or audio recorder 422, above-eye display module 424, headgear camera 28, or light sources 428 on the surgical display headgear 10.

Continuing with FIG. 9, headgear controller 26 is in communication with surgical tool controller 60 and both controllers are optionally connected to CPU 430 and external display 432. Through signals from headgear controller 26 to surgical tool controller 60, a user may access and control in-situ camera 64, sensors 436, light source 438, thermocouple 440, or other surgical tool components such as positioning circuitry 442. Other functions which are desirable for the control of a surgical tool such as power, temperature or time settings may also be linked to and controlled by surgical tool controller 60.

Continuing with the block diagram of FIG. 9, surgical tool controller 60, connected wirelessly (or wired depending on the embodiment) to headgear controller 26, transmits real-time images and sensor readings from in-situ camera 64 and sensors 436 which may then be displayed on above-eye display module 154, CPU 430 or other external display 432. Images from in-situ camera 64 or headgear camera 28, readings from sensors 436 and thermocouple 440 may be sent to CPU 430 for processing or storage in an adjacent memory location and/or to be used in combination with each other. For example, data from sensor 436 may be overlaid with images from in-situ camera 64 based on their axial or positional relationship to provide a map, for example a thermal map, of sensor readings on the surgical image which may then be displayed to above-eye display module 154 or to external display 432. In another example, the surgeon may activate headgear camera 28 to take selective still images of the surgical site from eye level while he or she is viewing moving video images on the above-eye display module 154 of the surgical process occurring at the site of operation as recorded by the in-situ camera and sensor system 58.

Continuing in reference to FIG. 9, Inputs to the surgical tool controller 60 and then to the headgear controller 26 may include visual images from in-situ camera 64, data from sensors 436, status of light sources 438 at the surgical tool, or data from a specific sensor such as thermocouple 440. In an aspect of the invention, surgical tool controller 26 may be programmed to send commands to each of these components as well. For example, surgical tool controller 26 may be programmed to automatically send on and off commands or specific commands to in-situ camera 64 to zoom, pan, focus, to record video or still images under specific conditions or based on input from sensors 436. Surgical tool controller 26 may also communicate with a remote device such as a cell phone 443 in another example.

Continuing with FIG. 9, surgical tool controller 26 is adapted to receive from and transmit signals to positioning circuitry 442 regarding the location of positioning circuitry 442 within a specific localized area of the patient's body. These signals may be utilized to determine the location of the enhanced surgical tool with respect to a reference map of interior portions of the patient's body as will be discussed in future paragraphs.

FIG. 10 illustrates a block diagram of another embodiment of the invention in which surgical display headgear 10 includes the same components as the embodiment of FIG. 9 including headgear controller 26 to receive input and send signals to control components of surgical display headgear 10. In the present embodiment, enhanced surgical tool 50 does not include a surgical tool controller 60. In the embodiment of FIG. 10, components of enhanced surgical tool 50 are in wireless, direct two-way communication with headgear controller 26. Wireless communication between components may utilize near field communication, Bluetooth, cellular, radio or other wireless communication technology. Headgear controller 26 is programmed to receive and control signals to and from enhanced surgical tool 50 components including in-situ camera 64, sensors 436, light source 438, thermocouple 440 and positioning circuitry 442. Each of these components are optionally in wireless communication with CPU 430 and external display 432 for display, processing, storage, analysis, and/or to be used in combination for further analysis such as for thermal mapping.

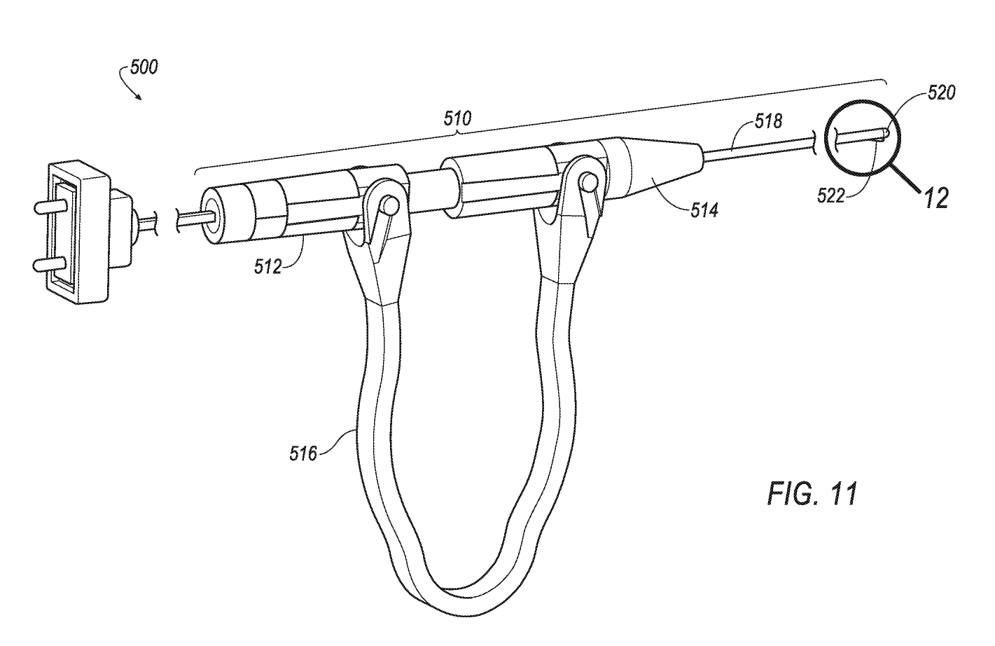

Shown in FIG. 11 is an embodiment of the invention including an enhanced surgical tool 500 which is in wireless communication with surgical display headgear 10 of FIG. 1. In this embodiment of the invention, enhanced surgical tool 500 includes a surgical tool 510 typical of an RF-powered surgical electrode such as the Elliquence Trigger-Flex surgical tool disclosed in U.S. Pat. No. 7,101,370 incorporated in its entirety herein by reference. It is understood that other surgical tools may be utilized in other embodiments of the invention. In this embodiment, surgical tool 510 is a hand-held surgical tool with body portions 512 and 514 interconnected with gripping mechanism 516, and including extended portion 518 to connect to active end 520 of the surgical tool 510. Active end 520 is directed toward the site of surgical operation by a person performing a surgical procedure. In this example, enhanced surgical tool 500 includes in-situ camera and sensor system 522 located adjacent the active end 520 of the surgical tool 510. In-situ camera and sensor system 522 includes an in-situ camera 524 capable of recording, photographing, video recording and transmitting visual information from the operational site via wireless communication to surgical display headgear 50 and to other external devices such as a computer or controller as discussed with respect to any of the previous embodiments. In-situ camera and sensor system 522 also includes sensors for sensing, collecting and transmitting characteristics of the surgical procedure. In-situ camera and sensor system 522 may also include positioning circuitry 442,

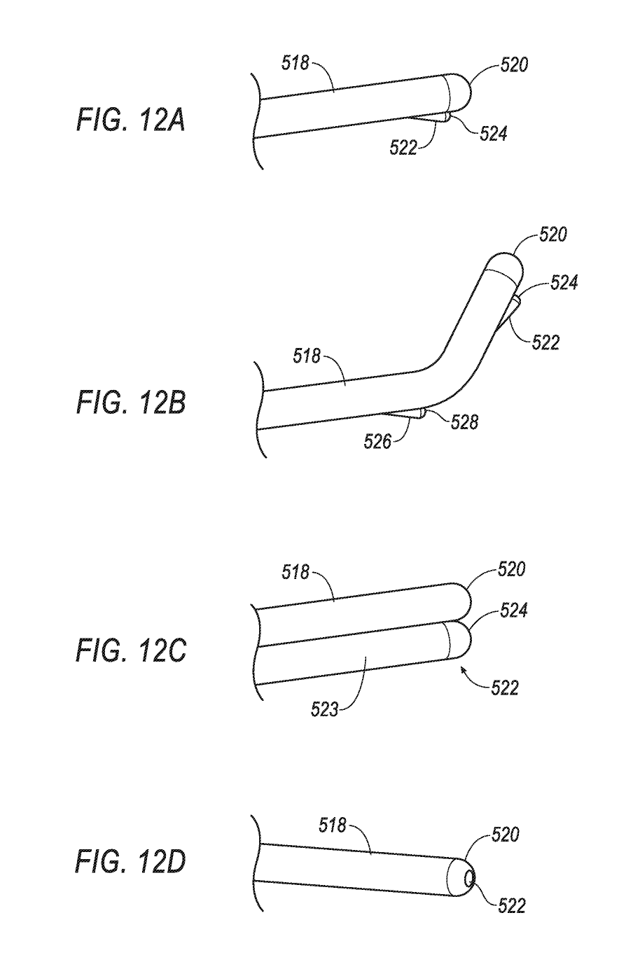

FIG. 12A and FIG. 12B include close-up perspective views of active end 520 of enhanced surgical tool 500. In the example of FIG. 12A, in-situ camera and sensor system 522 is located adjacent the active tip 520 of the surgical tool 510 positioned on the lower surface of extension portion 518. Forward-facing side 524 of in-situ camera and sensor system includes camera lens and the sensing ends of sensors. FIG. 12B illustrates an embodiment in which a second in-situ camera and sensor system 526 is located on the lower surface of the extension portion 518 closer to the gripping mechanism 516 and adjacent a bendable section 530 of extension portion 518. Forward-facing side 528 of in-situ camera and sensor system 526 includes camera lens and sensing end of sensors to be positioned to photograph or video record activity and collect sensor data from the surgical tool and body tissue at or very near the site of surgical operation. In-situ camera and sensor systems 522 and 528 can be located on other parts of the surgical tool 510 in locations that are further or closer to the active portion of surgical tool 520. When the user of the surgical tool 510 manipulates the active end 520 of the tool to perform a surgical procedure, the lens and sensors of camera and sensor system 522 are located in close proximity to the surgical procedure to transmit visual and sensor signals from the surgical site to surgical display headgear 10 which receives the signals and displays the transmitted images to above-eye display module 154. Through images recorded by in-situ camera and sensor system 522, the surgeon has a close-up view of the surgical process which then provides the close-up images to the above-eye display module 154 which located in an easily accessible vantage point of the surgeon's field of view.

Referring now to FIG. 12C, another embodiment of the present invention is shown and described. In FIG. 12C, camera extension 523 is shown positioned on extension portion 518 of the previous embodiment. Camera and sensor system 522 are positioned at a distal end of the camera extension 523 with respect to the body of the surgical device. The camera and sensor system 522 may be of any type described in previous embodiments and, in one aspect, communicates with surgical tool controller 60 through the camera extension 523. In operation, the camera extension 523 may expand, contract, or bend with the extension portion 518 or in connection with any of the electrodes described any of the patents incorporated herein by reference.

Referring now to FIG. 12D, another embodiment of the present invention is shown and described. In FIG. 12D, camera and sensor system 522 is shown embedded in active end 520. In this embodiment, the camera and sensor system 522 is insulated through an insulation layer from the electrode encompassing the active end 520. Camera and sensor system 522 may communicate with surgical tool controller 60 either wirelessly or through a communication electrode, fiber cable or conduit inside of the extension portion 518 or inside the electrode.

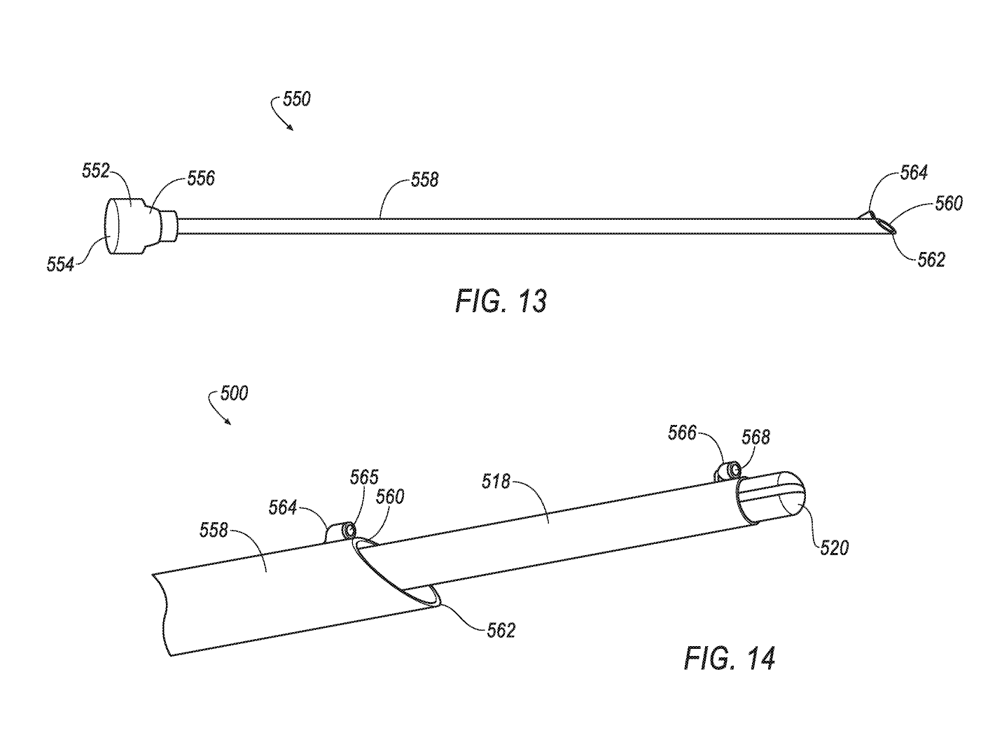

Referring now to FIG. 13 another embodiment of the present invention is shown and described which includes guide wire apparatus 550 for guiding the insertion of the active end of a surgical tool (shown in FIG. 11) into a site of surgical operation to a specific surgical site. Guide wire apparatus 550 includes entrance channel 552 with a broad opening 554 which connects to tapered portion 556 which tapers down to meet guide wire extension portion 558. Guide wire extension portion 558 leads to pointed tapered exit 560. Guide wire in-situ camera and sensor system 564 is situated on the outer surface of guide wire extension portion 558 and adjacent pointed tapered exit 560 and is positioned to record and measure actives at the surgical site. In this embodiment, guide wire in-situ camera and sensor system 564 is positioned above pointed tapered exit 560, but it could be located on a lower surface of pointed tapered exit opening 560 or on another part of guidewire apparatus 550.

FIG. 14 illustrates a perspective of a close-up view of pointed tapered exit 560 of guide wire apparatus 550 in which a portion of enhanced surgical tool 520 such as extension portion 518 a surgical tool such as Elliquence Trigger-Hex of FIG. 11 is positioned inside the guide wire extension portion 558. Active end 520 of surgical tool 520 extends outside of the pointed tapered exit 560 of guide wire apparatus 550 and includes in-situ camera and sensor system-sensor 566 located on an upper surface of extension portion 518 of enhanced surgical tool 520. In-situ camera 568 of camera and sensor system 566 includes lens 568 directed toward active end 520 of surgical tool 520. Additionally, guide wire in-situ camera and sensor system 564 is positioned on the upper surface of pointed tapered exit 560. In this example, guide wire in-situ camera 565 is positioned to face toward the portion of the enhanced surgical tool extending out of pointed tapered exit 560 for another perspective the activities of the site of surgery.

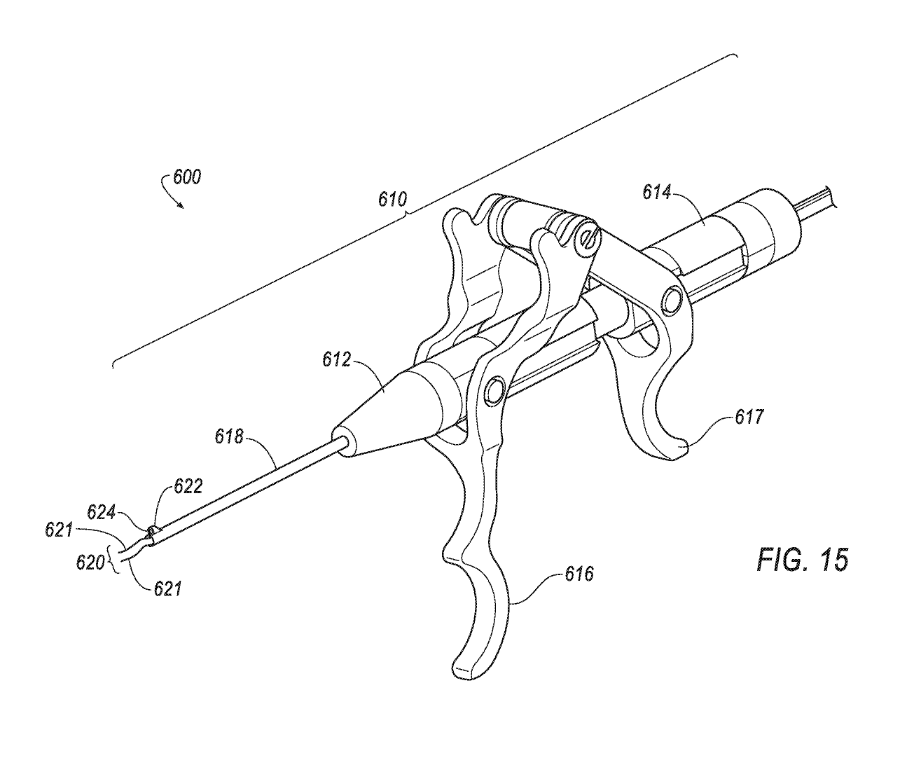

Referring now to FIG. 15, an embodiment of the invention is illustrated which demonstrates an enhanced surgical tool 600 which includes surgical tool 610 which, in this embodiment, may be a surgical tool similar to that disclosed in U.S. Pat. No. 7,905,882 incorporated in its entirety by reference. Enhanced surgical tool 600 includes an active end 620 which may include apparatus for surgical procedures such as electrode ends 621 for cutting or coagulating tissue. In-situ camera and sensor system sensor system 622 incorporated into surgical tool 610 near active end 620 includes in-situ camera lens 624 focused toward the specific tissue to receive the surgical activity. The in-situ camera and sensor system sensor system 622 is positioned to take close-up images, still or video recording, and to collect sensor data which are then transmitted to surgical display headgear 10 which receives the signals and displays the transmitted images to above-eye display module 154 or other display. It will be understood that the embodiments associated with FIGS. 12C and 12D may be used with the present embodiment.

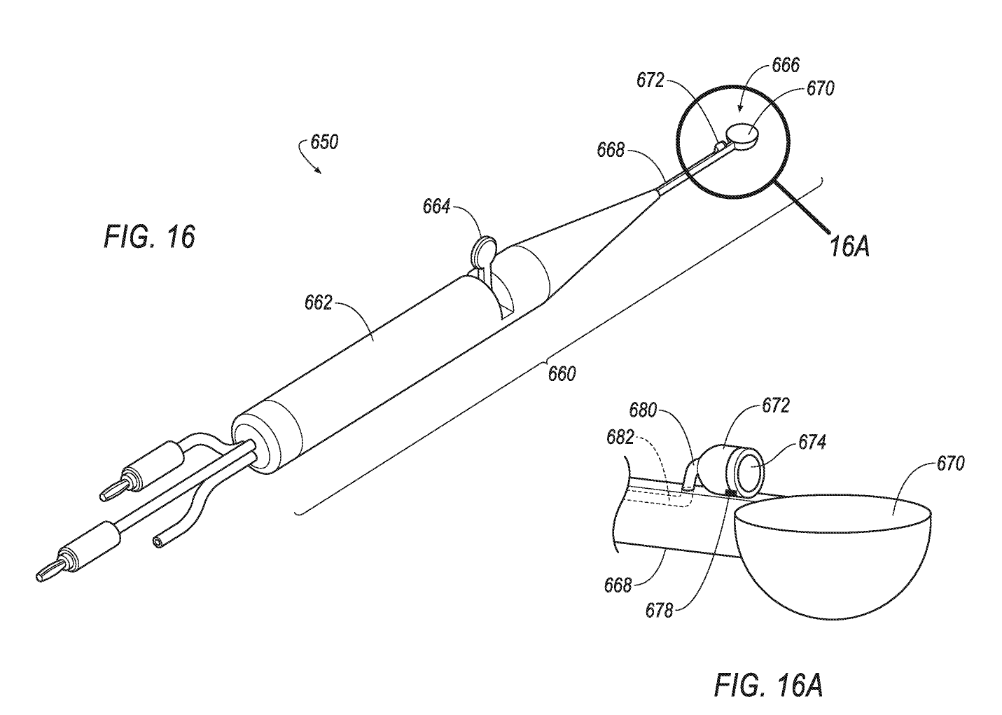

FIG. 16 demonstrates an embodiment of enhanced surgical tool 650 including surgical tool 660 as disclosed in U.S. Pat. No. 7,137,982 incorporated in its entirety by reference. Enhanced surgical tool 650 includes an extension portion 668 which may connect to an apparatus for surgical procedures such as semi-spherical scoop 670 at active end 666. Enhanced surgical tool 650 includes a slender hand piece 662 for a user's fingers and palm to hold enhanced surgical tool 650 and a switch 664 activated by a user's thumb. In-situ camera and sensor system 672 incorporated into enhanced surgical tool 650 near active end 666 includes in-situ camera lens 674 focused toward the specific tissue to receive the surgical activity. The in-situ camera and sensor system sensor system 672 is positioned to collect images, and still or video recording utilizing in-situ camera 674 and to collect sensor data from sensor 678, which are then transmitted to surgical display headgear 10 such as in FIG. 1, which receives the signals and displays the transmitted images to above-eye display module 152 or other display.

FIG. 16A illustrates a close-up perspective of an embodiment of the invention in which active end 666 of enhanced surgical tool 650 in which in-situ camera and sensor system 672 includes connection cable 680 which may be enclosed in a conduit or other protective structure. Connection cable 680 connects to enclosed conduit 682 to connect in-situ camera and sensor system to a surgical tool controller which is not shown in this illustration. A surgical tool controller in this example may be built into the hand piece 662 of the enhanced surgical tool. 650.

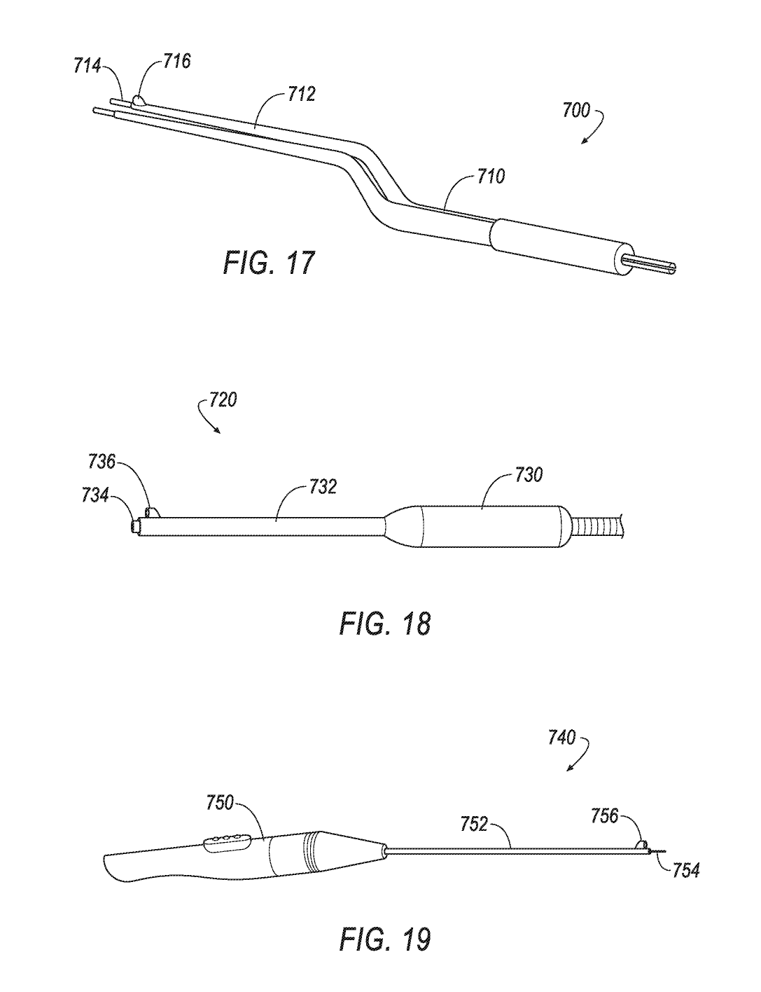

FIG. 17 demonstrates an embodiment of enhanced surgical tool 700 which includes electrode type forceps which may be a surgical tool similar to Elliquence Bipolar forceps. Enhanced surgical tool 700 includes gripping portion 710 by which a user may hold and maneuver enhanced surgical tool 700 during a surgical procedure. Gripping portion 710 is connected to extension portion 712 which connects to active end 714, the portion of enhanced surgical tool 700 closest to the site of a surgical operation during use. In-situ camera and sensor system 716 is incorporated into enhanced surgical tool 700 near active end 716 may include camera, sensors, positioning circuitry, and light sources (not specifically shown) In-situ camera and sensor system sensor system 716 is positioned to take images, still or video recording, collect sensor data, and control light sources at the surgical site which are then transmitted to surgical display headgear 10 such as that of FIG. 1 which receives the signals and displays the transmitted images to above-eye display module 154 or other display.

FIG. 18 demonstrates an embodiment of enhanced surgical tool 720 which includes suction-type surgical tool similar to Elliquence suction coagulator. Portion 730, by which a user may hold and maneuver enhanced surgical tool 720 during a surgical procedure is connected to extension portion 732 which connects to active end 734, the portion of enhanced surgical tool 720 closest to the site of a surgical operation during use. In-situ camera and sensor system 736 incorporated into enhanced surgical tool 720 near active end 734 may include camera, sensors, and light sources (not specifically shown) In-situ camera and sensor system sensor system 736 is positioned to take images, still or video recording, collect sensor data, and control light sources at the surgical site which are then transmitted to surgical display headgear 10 which receives the signals and displays the transmitted images to above-eye display module 154 or other display.

FIG. 19 demonstrates an embodiment of enhanced surgical tool 740 which includes electrode-type surgical tool as disclosed in U.S. patent application Ser. No. 14/207,990 incorporated in its entirety by reference. Portion 750 is connected to extension portion 752 which connects to active end 754, the portion of enhanced surgical tool 740 closest to the site of a surgical operation during use. In-situ camera and sensor system 756 incorporated into enhanced surgical tool 740 near active end 754 may include camera, sensors, positioning circuitry, and light sources (not specifically shown) In-situ camera and sensor system sensor system 756 is positioned to take images, still or video recording, collect sensor data, and control light sources at the surgical site which are then transmitted to surgical display headgear 10 such as that of FIG. 1 which receives the signals and displays the transmitted images to above-eye display module 154 or other display.

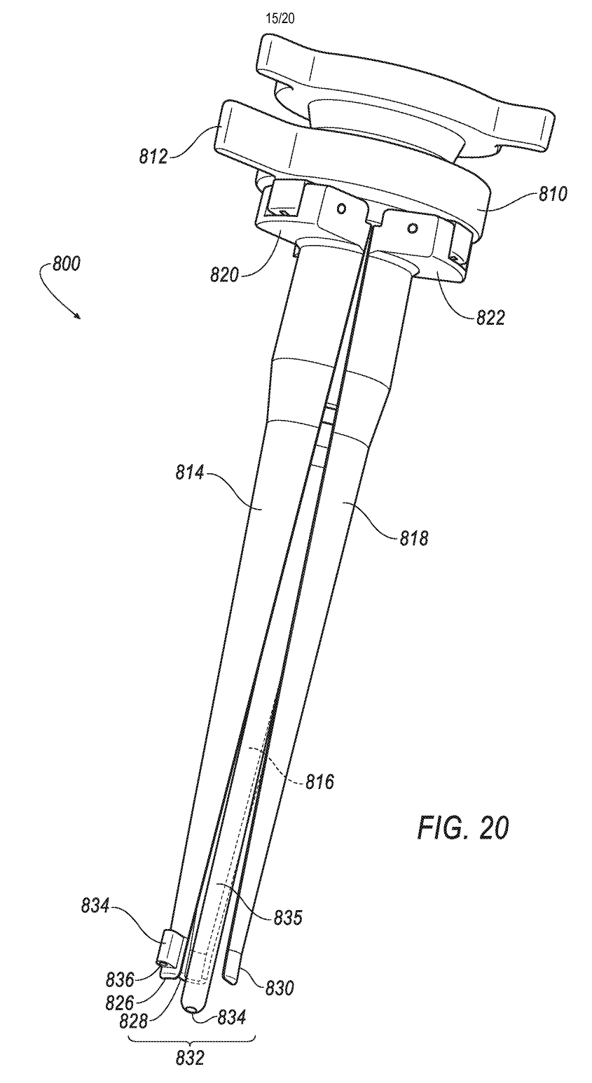

FIG. 20 illustrates an embodiment of enhanced surgical tool 800 which includes a tissue retractor type of surgical tool as disclosed in U.S. patent application Ser. No. 13/899,492 incorporated in its entirety by reference. Enhanced surgical tool 800 includes retractor blades 814, 816, and 818 which extend outward and end in blade tips 826, 828 and 830 to form an active end 832 In-situ camera and sensor system 834 incorporated into enhanced surgical tool 800 on the outside of blade tip 826 and near active end 832 may include camera, sensors, positioning circuitry, and light sources. (not specifically shown) In-situ camera and sensor system sensor system 834 is positioned to take images, still or video recording, collect sensor data, and control light sources at the surgical site which are then transmitted to surgical display headgear 10 such as that of FIG. 1 which receives the signals and displays the transmitted images to above-eye display module 154 or other display. In-situ camera and sensor system 834 may also be positioned on an inside of the blades 814, 816 and 818 adjacent active end 832 or system 834 may be positioned on an inside of the blades 814, 816 and 818 closer to hinge apparatus 820 and 822. Also, in-situ camera and sensor system 834 is shown attached to camera shaft 835 that extends down inside the blades 814, 816, and 818 as described with respect to a dilator or other device used in connection with the retractor disclosed in the aforementioned patent application.

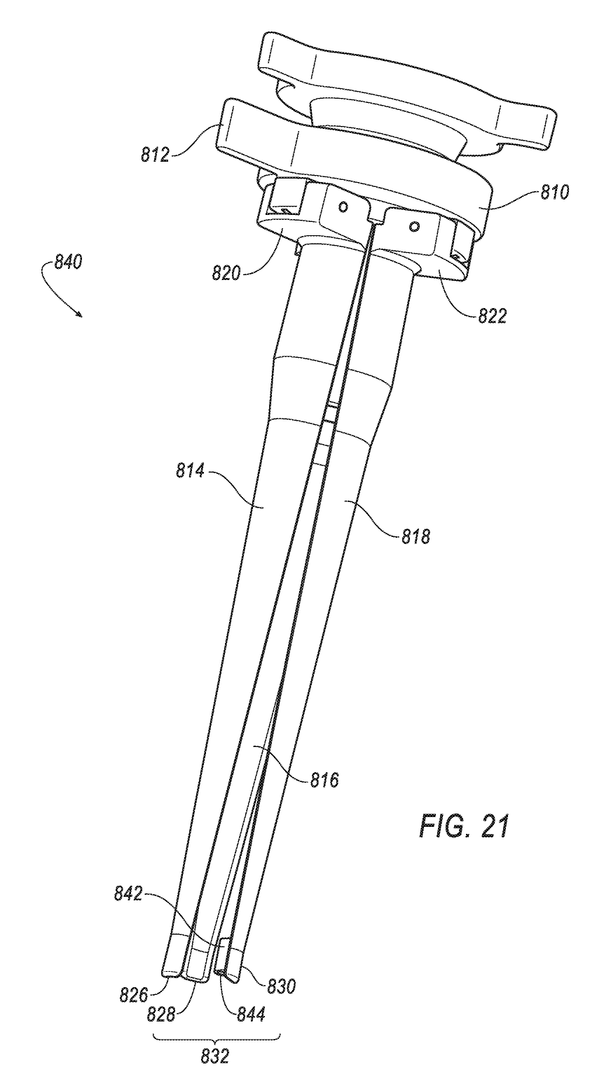

FIG. 21 illustrates another embodiment of the invention with enhanced surgical tool 840 utilizing a tissue retractor type surgical tool as disclosed in U.S. patent application Ser. No. 13/899,492 incorporated in its entirety by reference. In this example, in-situ camera and sensor system sensor system 842 is positioned on the inside surface of blade tip 830 near active end 832 of enhanced surgical tool 840.

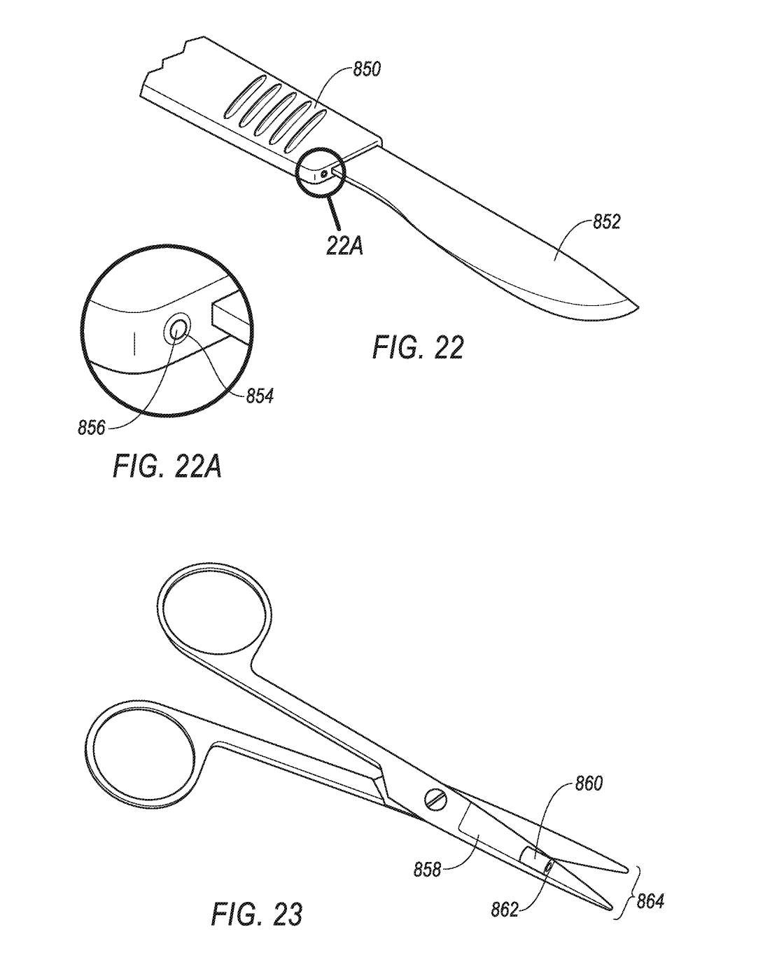

FIG. 22 illustrates an embodiment of the invention in which in-situ camera and sensor system 854 is built into handle 850 of a scalpel type surgical tool where the lens and sensor face 856 are directed toward the scalpel blade 852.

FIG. 22A includes a close-up of the in-situ camera embedded within the aforementioned scalpel type surgical tool including sensor system sensor system 854 and sensor face 856.

FIG. 23 illustrates an embodiment of the invention in which in-situ camera and sensor system sensor system 860 is built into blade 858 of a scissor type surgical tool where the lens and sensor face 862 are directed toward active end 864 of the scissors.

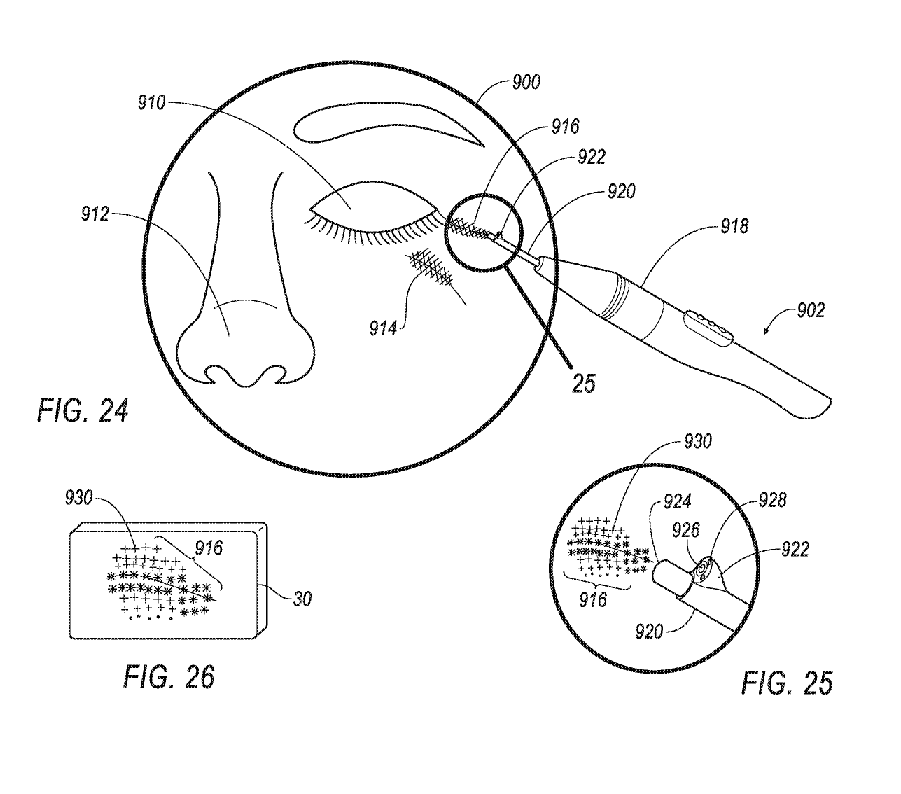

FIG. 24 illustrates an embodiment of the operation of the invention in which enhanced surgical tool 902 is utilized to affect an area tissue on a portion of a face 900. An eye 910 and a nose 912 are included as a reference point for body tissue portions 914 and 916 on the face 900 being surgically modified. In this example, enhanced surgical tool 902 may be a heat producing surgical tool such as an electrode and which includes gripping portion 918 connected to extension portion 920 which is connected to active end 924 as shown in FIG. 25. An outer surface of extension portion 920 which, in this example, is cylindrically-shaped includes in-situ camera and sensor system 922 adjacent active end 924. Lens of in-situ camera 926 and sensor input end 928 of in-situ camera and sensor system 922 are positioned to face active end 924. In-situ camera 926 records images of the surgical site and sensor 928 collects sensor data such as temperature and other characteristics and each transmit this information to, in one example of the invention, headgear controller 26 as shown in FIG. 1. In one aspect with respect to FIG. 10, sensors 436 and camera 64 provide visual temperature information back to headgear controller 26 shown in FIG. 1. Headgear controller 26 processes the received information and provides it to above eye display 154 in the form of the thermal map 930 as shown in FIG. 25. Accordingly, the image provided by headgear controller 26 to above-eye display 154 includes the thermal map 930 provided by the sensors 436 (such as infrared sensors that detect heat and processed into an image by headgear controller 26) overlaid on top of a visual image provided by the camera 64. As the positioning of the sensors and the camera are co-located in the same body, the sensors and camera may have a known relationship there between to permit the headgear controller 26 to correlate the images such that they are overlaid. In FIG. 25, a thermal map 930 showing a gradient of darker and lighter dots or pixels or colors may be overlaid onto an image of portion of tissue 916 at the surgical site to provide a thermal map 930 showing the gradients of temperature at different locations on the tissue 916. Headgear controller 26 of FIG. 1 transmits the thermal map 930 for display in the above-eye display module 154. The combination of thermal measured data from sensors 928 and visual images from camera 926 can assist the surgeon in understanding the specific conditions existing at the location of the active end 924 of enhanced surgical tool 902 and the tissue portion 916.

Readings or data collected through a sensor or images collected through a camera during a surgical procedure may be used to characterize the conditions of body tissues and their composition. For example, enhanced surgical tool 902 of FIG. 24 may include a number of sensors 928 (included in any of the previously described embodiments of in-situ camera 926) for detecting characteristics such as tissue temperature, inductive properties, and moisture levels, and visual indicators such as color or texture. As enhanced surgical tool 902 moves through a particular portion of diseased tissue, the characteristics and conditions of this diseased tissue are collected and may be uniquely characterized based on the sensor readings of sensor 928 and visual images from in-situ camera 926. As the enhanced surgical tool 902 moves from diseased tissue to a particular portion of healthy tissue, the characteristics and conditions of the healthy tissue are also collected and characterized. Particular readings of sensor data and/or visual images may indicate to a surgeon when diseased tissue has been removed and the enhanced surgical tool 902 has arrived at a location of healthy tissue and to end the surgical process. In this way surgeon can be more selective, affecting only the diseased tissue, and minimizing the surgical impact on healthy tissue. If the enhanced surgical tool 902 performs an active procedure on tissue 916 such as applying heat or RF power for cutting or coagulating tissue, sensor 928 can also detect tissue conditions in response to the specific tool settings of enhanced surgical tool 902, for example, to automatically adjust enhanced surgical tool 902 when the temperature is too hot or too cold.

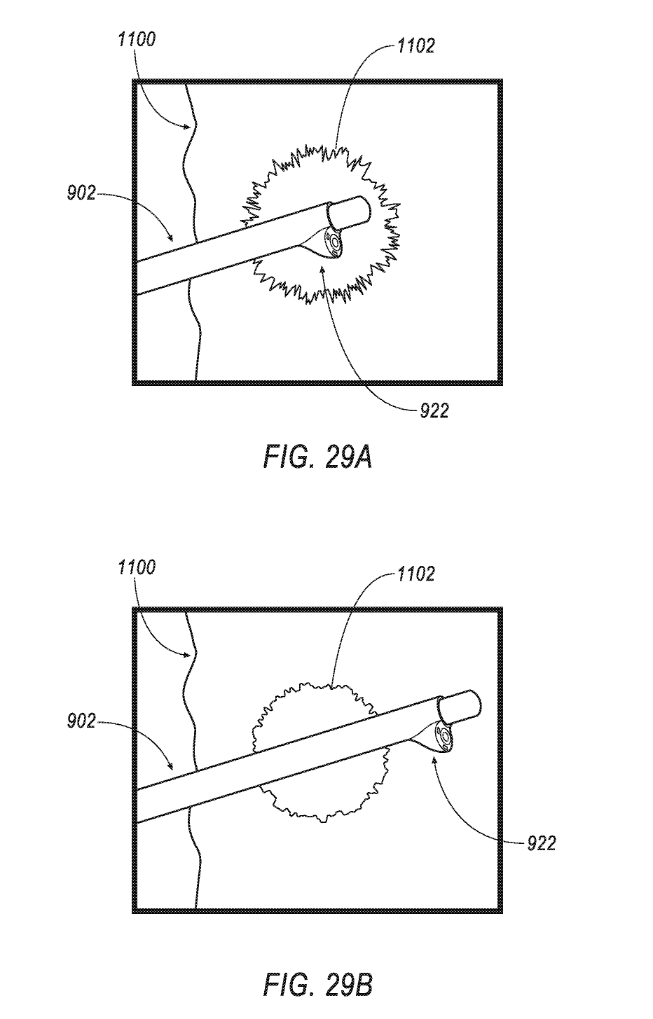

In one example, as shown in FIGS. 29A and 29B, enhanced surgical tool 902 is shown being inserted into tissue 1100. The tip of enhanced surgical tool 902 includes camera and sensor system 922 as described in any of the previous embodiments. As shown in FIG. 29A, camera and sensor system 922 is positioned within diseased tissue 1102. Diseased tissue 1102 may have particular characteristics such as decreased moisture, increased temperature and other attributes that may be detected by camera and sensor system 922. Such measured characteristics, referring to FIG. 10 for example, may be provided by sensors (436 in FIG. 10) back to headgear controller 26. In reference to FIG. 9, headgear controller 26 then provides information to the surgeon through above eye display 424 such as by way of temperature or moisture indicators displayed in the above eye display 424. Through this, the surgeon using the present system can determine that the tip of enhanced surgical tool 902 is currently positioned within diseased tissue 1102.

With respect to FIG. 29B, camera and sensor system 922 is now located outside of diseased tissue 1102 and back within tissue 1100. Similar to the information flow described above, the surgeon will be provided with the needed information that indicates enhanced surgical tool 902 is now located outside the scope of the diseased tissue 1102.

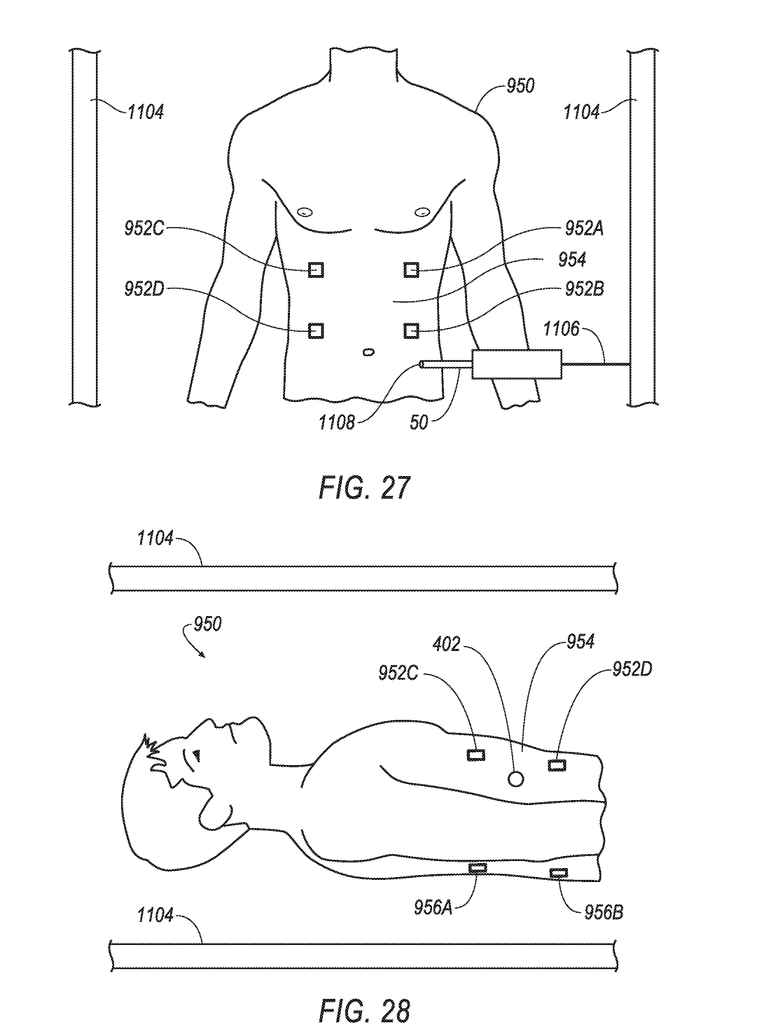

Now referring to FIG. 27, a top view perspective of a patient's torso is illustrated according to an aspect of the invention which describes a positioning system to detect the position of an enhanced surgical tool within a patient's body 950. In this example, reference markers 952A through 952D are attached to torso 950 of a patient's body 950 to provide reference points for providing a positional map of the interior portions of the patient's body. FIG. 28 illustrates the invention from a side view of a patient's body 950 including reference marker 952C, 952D and 956A and 956B demonstrating exemplary locations of reference markers on the upper and lower sides of a patient's torso 950. This example would be applicable for a surgical site located in a portion of the patient's body such that reference markers 952A-D and 956A-B are spatially distributed around the surgical site and surround the surgical site in three dimensions. The reference markers 952A-D and 956A-B are utilized in conjunction with positioning circuitry 442, located in camera and sensor system of enhanced surgical tool 50 as illustrated in FIG. 9, to provide an electrical signal indicating the location of the enhanced surgical tool 50 in a 3-dimensional space, and more particularly, with respect to the reference markers 952A-952D and 956A-B.

The reference markers 952A through 952D and 956A and 956B are applied to the surface of a patient's skin. It should be understood that additional or fewer markers may be applied in other embodiments of the invention. The reference markers 952A-D and 956A-B may be comprised of non-magnetic metal, such as lead or phosphorescent tags so that the location of the reference marker will be detected in an x-ray, CT scan, MRI, ultrasound or other scanning instrument 1104 which can provide visual detail of the interior of the patient's body. In another embodiment, scanning instrument 1104 includes receiving apparatus such that it receives signals reflected from any of the markers. This received signal is then sent to a CPU 430 (see FIG. 9) such as that contained in enhanced surgical tool 50. The reference markers may be of different shape, size, location, or frequency than is shown in FIGS. 27 and 28 and may also include sensing and response circuitry to be utilized in a positioning system. The reference markers 952A-D and 956A-B are placed on a patient's body 950 in strategic positions in order to provide a three dimensional set of reference points around a site of surgical operation. Reference markers 952 A through D and 956A and 956B may be pre-positioned on an adhesive cloth or layer with is then attached to a patient's body like an adhesive tape. The tape may include electrical connections between the reference markers 952A-D and 956A-B. Additionally, an isotope marker may be implanted in strategic locations of the patient's body 950 to provide internal reference points which will be detected in a scan of the patient's body 950.

A scan of the body is performed to produce a 3-dimensional, computerized, geographical, map which for this invention is referred to as a body reference map. A body reference map is a map of the interior structures of the patient's body as determined from the scan such as internal organs and bones and their location with respect to the reference markers and isotope markers. The body reference map is stored in memory for future reference. In reference to FIG. 9, the memory location may be in headgear controller 26, surgical tool controller 60, or an external controller or computer such as CPU 430 and is accessible in real time.

An example of a method of use of an embodiment of the invention of FIGS. 27 and 28 is now described. A surgical procedure is performed using enhanced surgical tool 50 which includes a positioning circuitry 442 (see FIG. 9) located on enhanced surgical tool 50 such as at the active end or distal end and adjacent to the portion of the enhanced surgical tool 50 inserted into the body. Reference markers 952A through 952D and 956A through 956B are attached to the patient's body in various locations surrounding the site of the surgical procedure to permit 3D mapping as described above. Scans of the patient's body are performed to create a body reference map of the interior of the patient's body with respect to the reference markers 952A through 952D and 956A through 956B and isotope markers and stored in memory. It will be understood that any type and number of markers may be used as desired to generate the 3D image.

During a surgical procedure, the positioning circuitry 442 (see FIG. 9) emits a real-time signal in the form of a pulse which is transmitted to reference markers 952A through 952D and 956A through 956B. The signal may be acoustic, electromagnetic, radio wave or other type of signal for transmitting position and which is received and reflected by the reference marker back to the positioning circuitry 442. In one example of the invention, the positioning circuitry 442 includes a control unit for measuring the timing of the return interval of each reflected signal and for calculating distances between reference markers and isotope markers and the positioning circuitry on the tip of the enhanced surgical tool 50. In another example, the reference markers 952A-D and 956A-B may include circuitry to actively sense, measure, and respond to signals transmitted from positioning circuitry 442. In another example, a control unit (not shown) for sensing the timing of signals between positioning circuitry 442 and reference markers 952A-D, 956A-B and for calculating distances is separate from the enhanced surgical tool 50 and is located in a separate sensor or set of sensors external to the patient body. Methods such as triangulation, trilateration, multilateration or time difference of arrival techniques may be used to determine the location of the positioning circuitry 442 relative to the reference markers 952A-D, 956A-B and isotope markers.

In another embodiment, enhanced surgical tool 50 includes a reflective tip 1108. The reflective tip 1108 may reflect any of the transmitted signals from the scanning instrument 1104. The reflected signals are received by the scanning instrument 1104 and, according to the above triangulation methods, the positioning circuitry 442 in the enhanced surgical tool 50 identifies the location of the markers, reflective tip and organs within the patient and provides this information via display the surgeon such that the surgeon may ascertain the location of the reflective tip 1108 of the enhanced surgical tool 50 with respect to organs inside the patient.

Once the position of the positioning circuitry 442 (see FIG. 9) relative to the reference markers 952A-D 956A-B is determined, it is tracked and continuously refreshed as the computer 430 or controller provides a compiled visual map of the real-time location and movement of the enhanced surgical tool 50 relative to the body reference map previously complied and stored in memory. The compiled visual map is projected for display onto above-eye display 154 or another external display 432.

In the previously described embodiment of the present invention, the surgeon may detect the position of body structures relative to enhanced surgical tool 50 in order to better navigate the enhanced surgical tool 50 of FIG. 2 within the body and will have visual images to indicate the location of specific body parts. For example, a tumor may be indicated as being in close relation to a delicate nerve area. In one example, marked with an isotope marker, the surgeon may use the positioning circuitry 442 to help detect how close the active end 56 of enhanced surgical tool 50 is to the nerve area. Boundaries can be established in the body reference map to provide alarms such as messaging to the above-eye display module 154 when the surgical tool is close to a sensitive area. Active end 56 of enhanced surgical tool 50 also includes sensors and a camera system 58 to provide real time information of operational characteristics from sensors and visual display from in-situ camera 64 which can also be overlaid and combined with the body reference map to provide a complete visual display of tool location for navigation, thermal mapping, infrared images, all overlaid into a 3-D image of the surgical site and displayed to above-eye display module 154.

In this specification, various preferred embodiments may have been described with reference to the accompanying drawings. It will, however, be evident that various modifications and changes may be made thereto, and additional embodiments may be implemented without departing from the broader scope of the invention as set forth in the claims that follow. The present invention is thus not to be interpreted as being limited to particular embodiments and the specification and drawings are to be regarded in an illustrative rather than restrictive sense.

It will be appreciated that the system and methods described herein have broad applications. The foregoing embodiments have been chosen and described in order to illustrate principles of the methods and apparatuses as well as some practical applications. The preceding description enables others skilled in the art to utilize methods and apparatuses in various embodiments and with various modifications as are suited to the particular use contemplated. In accordance with the provisions of the patent statutes, the principles and modes of operation of this invention have been explained and illustrated in exemplary embodiments.

It is intended that the scope of the present methods and apparatuses be defined by the following claims. However, it must be understood that this invention may be practiced otherwise than what is specifically explained and illustrated without departing from its spirit or scope. It should be understood by those skilled in the art that various alternatives to the embodiments described herein may be employed in practicing the claims without departing from the spirit and scope as defined in the following claims. The scope of the invention should be determined, not with reference to the above description, but should instead be determined with reference to the appended claims, along with the full scope of equivalents to which such claims are entitled. It is anticipated and intended that future developments will occur in the arts discussed herein, and that the disclosed systems and methods will be incorporated into such future examples. Furthermore, all terms used in the claims are intended to be given their broadest reasonable constructions and their ordinary meanings as understood by those skilled in the art unless an explicit indication to the contrary is made herein. In particular, use of the singular articles such as “a,” “the,” “said,” etc. should be read to recite one or more of the indicated elements unless a claim recites an explicit limitation to the contrary. It is intended that the following claims define the scope of the invention and that the method and apparatus within the scope of these claims and their equivalents be covered thereby. In sum, it should be understood that the invention is capable of modification and variation and is limited only by the following claims.

What is claimed is:

1. An enhanced surgical tool and display device for conducting surgical procedures, comprising:
a wearable headgear including a frame for positioning an optical display into a field of view;
a controller attached to a portion of the frame, said controller adapted to provide a computer generated image to said optical display;
the optical display including an active side adapted for projecting the computer generated image, wherein said optical display is in electrical communication with the controller, and wherein said optical display is attached to the controller such that the active side is visible in a field of view;
a surgical tool including a first end for grasping the surgical tool and a second distal end for surgical contact with a region of surgically operative material; and
a camera attached to the second distal end of the surgical tool and in wireless communication with the controller, wherein said camera is adapted to record and transmit visual images from a region of surgically operative material adjacent the distal end of the surgical tool to the controller, and wherein said controller is adapted to configure the visual images for projection to the active side of the optical display.

2. The enhanced surgical tool and display device according to claim 1, wherein the camera is adapted to record a video image and a still image and wherein the controller is adapted to provide an immediate projection of the image to the optical display.

3. The enhanced surgical tool and display device according to claim 2, further comprising
at least one sensor positioned at the distal end of the surgical tool and in wireless communication with the controller; and
wherein said sensor is adapted for measuring at least one operational characteristic from the region of surgically operative material adjacent the distal end of the surgical tool.

4. The enhanced surgical tool and display device according to claim 3, wherein the sensor is adapted to transmit an electrical signal to the controller and wherein the controller provides a visual image related to the signal transmitted from the sensor to the optical display.

5. The enhanced surgical tool and display device according to claim 4, wherein the operational characteristic measured at the sensor is one of a member of a set consisting of hydration of body tissue, calcification of body tissue, power level, power mode, power usage, amperage, voltage, temperature, resistance, liquid flow rate, and time.

6. The enhanced surgical tool and display device according to claim 5, wherein the sensor is a retractable thermocouple.

7. The enhanced surgical tool and display device according to claim 6 wherein the controller further comprises:
a first memory component adapted to receive and store an electrical signal transmitted by the sensor relating to the operational characteristic from the region of surgical material from adjacent the distal end of the surgical tool;
a second memory component adapted to receive and store the visual image transmitted by the camera of the region of surgical material from adjacent the distal end of the surgical tool;
wherein the controller is adapted to provide the optical display which combines a sensor signal and the visual image from the camera in axial relationship.

8. An enhanced surgical tool for conducting a surgical procedure, comprising:
a wearable headgear including a frame for positioning an optical display into a field of view;
a controller attached to a portion of the frame, said controller adapted to provide a computer generated image to an optical display;
the optical display including an active side adapted for projecting the computer generated image, wherein said optical display is in electrical communication with the controller, and wherein said optical display is attached to the controller such that the active side is visible in a field of view;
a surgical tool including a first end for grasping the tool and a second distal end for surgical contact with a region of surgically operative material;
a sensor system attached to the second distal end of the surgical tool and in electrical communication with the controller, wherein said sensor system is adapted to transmit location information of the distal end to the controller;
a plurality of reference markers distributable about a patient's body and in electrical communication with the controller, wherein the plurality of reference markers provides three dimensional position information to the controller;
wherein said controller is adapted to configure a location of the distal end with respect to the three dimensional position information.

9. The enhanced surgical tool according to claim 8, further comprising:
image data providing location information for at least one internal bodily structure in the patient's body, wherein the image data is provided to the controller;
wherein the controller is adapted to configure the location of the second distal end with respect to the at least one internal bodily structure.

10. The enhanced surgical tool according to claim 9, wherein:
the internal bodily structures include at least a spine; and
the location of the second distal end is provided with respect to the location of the spine.

11. The enhanced surgical tool according to claim 10, wherein the image data is provided by an X-ray or MRI machine.
